(19) United States
(12) Patent Application Publication
(10) Pub. No.: US 2015/0012042 A1
(43) Pub. Date: Jan. 8, 2015

(54) ORTHOPEDIC IMPLANTATION DEVICE

(71) Applicant: Institute for Musculoskeletal Science and Education, Ltd., Radnor, PA (US)

(72) Inventor: Michael Black, Phoenixville, PA (US)

(73) Assignee: Institute for Musculoskeletal Science and Education, Ltd.

(21) Appl. No.: 14/321,454

(22) Filed: Jul. 1, 2014

Related U.S. Application Data
(60) Provisional application No. 61/842,993, filed on Jul. 4, 2013.

(51) Int. Cl. A61B 17/70

(52) U.S. Cl. CPC A61B 17/7032
USPC 606/269

(57) ABSTRACT

An orthopedic implantation device that includes tulip element, a coupling wedge and a locking assembly, and a kit and method of use thereof are described herein. The coupling wedge is located in the bottom of the tulip element. The tulip element includes transverse slots for receiving the rod therein until the rod sits on the coupling wedge. The locking assembly includes a locking cap adapted to be received completely within the tulip element, and having flanges and tabs that cooperate with corresponding grooves in the tulip element. The locking assembly includes a cap set screw adapted to be received within a thru hole in the locking cap element.

### CROSS REFERENCE TO RELATED APPLICATIONS

This application claims priority to U.S. Provisional Application Ser. No. 61/842,993, filed Jul. 4, 2013, the disclosure of which is incorporated herein by reference in its entirety.

### FIELD OF THE INVENTION

The present invention relates to orthopedic implantable devices, more particularly, pedicle screw and rod systems for joining two or more bone segments, such as vertebrae.

### BACKGROUND OF THE INVENTION

Bone screws are utilized in many types of spinal surgery in order to secure various implants to vertebrae along the spinal column for the purpose of stabilizing and/or adjusting spinal alignment. Although both closed-ended and open-ended bone screws are known, open-ended screws are particularly welt suited for connections to rods and connector arms because such rods or arms do not need to pass through a closed bore, but rather can be laid or urged into an open channel within a receiver or head of such a screw.

A common mechanism for providing vertebral support is to implant bone screws into certain bones which in turn support or are supported by a longitudinal structure, such as a rod. Bone screws of this type may have a fixed head or receiver relative to a shank thereof. In the fixed bone screws, the rod receiver head cannot be moved relative to the shank, and the rod must be favorably positioned in order for it to be placed within the receiver head. This is sometimes difficult or impossible to do. Therefore, polyaxial bone screws are commonly preferred.

Typical open-ended bone screws include a threaded shank with a pair of parallel projecting branches or arms, which form a yoke with a U-shaped slot or channel to receive a rod. Hooks and other types of connectors used in spinal fixation techniques may also include open ends for receiving portions of rods or other longitudinal structures.

During the rod implantation process it is desirable to utilize bone screws or other bone anchors that have components, or inserts that remain properly aligned throughout the implantation process. It is also desirable for the components to be easily assembled, without increasing the complexity or number of steps required during implantation.

Therefore it is an object of the present invention to provide improved pedicle screws that can be used with a rod system.

It is a further object to provide improved orthopedic fixation devices.

It is yet a further object to provide an improved method for joining two or more bone segments together.

### SUMMARY OF THE INVENTION

Orthopedic implantation devices, specifically, pedicle screws, kits, and methods for joining two or more bone segments with these devices are described. herein. The devices contain a bone fastener, a coupling element which includes a coupling wedge and a tulip element, and a locking means, such as a set screw.

The bone fastener can include a pedicle screw which includes a first end, a threaded intermediate portion, and a second end, preferably in the form of a screw head. The screw head may have a roughened side surface. In some embodiments the side surface contains threads (or grooves) that are parallel to each other. In preferred embodiments, the roughened surface includes patterned grooves. Patterned grooves allow for increased locking strength. The threaded intermediate portion preferably has a dual thread lead. The first end of the screw preferably contains a blunt tip.

The coupling wedge is configured to be received completely in the tulip element, and contains an upper portion, a lower portion and a thru hole. The lower portion of the coupling wedge has an inner surface, which may be shaped as a concave approximately spherical or ball-shaped, segment. The inner surface is preferably a concave spherical cavity. In some embodiments, the inner surface of the coupling wedge is adapted to engage the head of the pedicle screw. In some embodiments, the inner surface is roughened, threaded, sandblasted, or includes a plurality of grooves. The upper portion of the coupling wedge has indentation adapted for receiving a rod and tabs for engaging the coupling element.

The locking cap has a generally cylindrical cap body, with an upper portion, a lower portion and a thru hole. The cap end is configured to be received completely in the tulip element and to rest completely within an interior recess in the tulip element. The cap preferably does not contain any protrusion that extends from the upper end of the upper portion and outside of the tulip element. The cap includes a plurality of radially spaced flanges and tabs extending radially outward from the lower portion of the cap. The upper portion of the locking cap element includes driver attachment pockets.

The tulip element includes an upper portion, an intermediate portion and a lower portion. The upper portion may define an interior recess which allows for assembly a rod into the tulip element. The tulip element is configured at its upper end to receiver the cap element completely, and it is configured at its intermediate portion to receive and orient the coupling wedge element.

Also described herein is a method of using the orthopedic implantation device described herein. In use, the bone screw is inserted from the top portion of the tulip, so that the head of the bone screw mates with the bottom of the tulip. The coupling wedge is pressed into the tulip from the top surface, until it locks into the position. This assembly accepts a rod, which is locked into place using the locking cap element and set screw.

### BRIEF DESCRIPTION OF THE DRAWINGS

FIGS. 1A and 1B are different views of the orthopedic fixation device described herein with a rod. FIG. 1A is an exploded view. FIG. 1B is an isometric view of the assembled orthopedic fixation device with a rod.

FIGS. 2A-2C are different views of the pedicle screw. FIG. 2A is a side elevation view of a pedicle screw. FIG. 2B is a top elevation view, showing the hexalobe drive on the head of the pedicle screw. FIG. 2C is a bottom elevation view, showing the dual lead thread on the shaft of the pedicle screw.

FIGS. 3A-3D are different views of the coupling wedge. FIG. 3A is an isometric view from the front, top, right of the coupling wedge. FIG. 3B is a front elevation view. FIG. 3C is a side elevation view. FIG. 3D is an isometric view of the coupling wedge in an upside down position, from the front, top, right of the coupling wedge.

FIGS. 4A-46 are different views of the locking cap, with and without the tulip element. FIG. 4A is a top perspective view of the locking cap. FIG. 4B is a top elevation view of the locking cap. FIG. 4C a top perspective view, showing the locking cap engaging the tulip element. FIG. 4D is a top 110 perspective view, partially in section, showing the locking cap in the tulip element in the unlocked position. FIG. 4E is a top perspective view, showing the locking cap turned such that it is between the locked and unlocked positions. FIG. 4F is a top perspective view, showing the locking cap in the tulip element, where the bench tabs exit the tulip bench tab tracks. FIG. 4G is a top elevation view of the locking cap and tulip in the unlocked position, as depicted in FIG. 4D.

FIG. 5A-5C show different views of the tulip element. FIG. 5A shows a top perspective view of the tulip element. FIG. 5B is a top elevation view of the tulip element. FIG. 5C is a side elevation view of the tulip element, showing its tapered reduction geometry.

FIGS. 6A-6C show different view of the pedicle screw with tulip. FIG. 6A is a cross-sectional view; FIG. 6B is a side elevation view; FIG. 6C is an isometric view from the front, top, right.

FIGS. 6D-6F show different views of an assembled orthopedic fixation device. FIG. 6D is a magnified, partial cross-sectional view of FIG. 6F; FIG. 6E is a cross-sectional view along line A-A of FIG. 6F. FIG. 6F is a side elevation view.

FIGS. 7A and 7B show different views of the locking set screw assembled in the locking cap thru hole. FIG. 7A is a cross-sectional view of section A-A in FIG. 7B. FIG. 7B is side elevation view of the locking set screw assembled in the locking cap.

### DETAILED DESCRIPTION OF THE INVENTION

### I. Orthopedic Fixation Devices

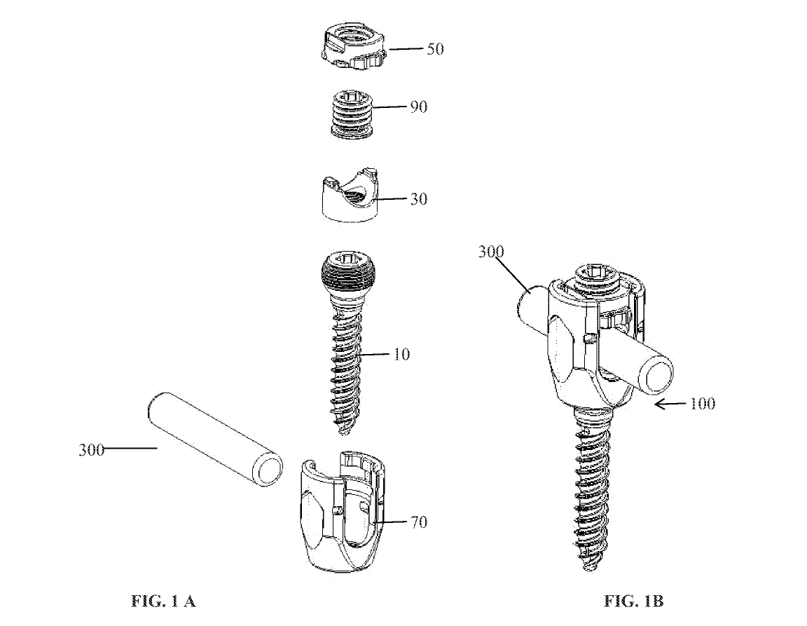

An orthopedic fixation device (100) as described herein is shown in FIG. 1A and 1B. The orthopedic fixation device (100) includes (i) a bone fastener (10), and a coupling element, which includes a coupling wedge (30), a locking cap (50), and a tulip element (70). The orthopedic fixation device (100) is assembled for use with a rod (300). The tulip element (70) is configured to receive all of the locking cap (50), the coupling wedge (30) and a rod (300). A locking set screw (90) secures the cap in the tulip element (70). The assembled orthopedic fixation device (100) with a rod (300) is illustrated in FIG 1B.

A. Bone Fastener

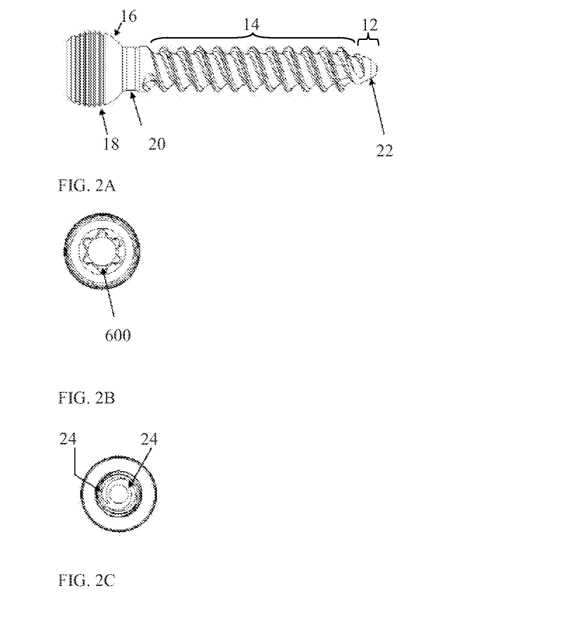

FIG. 2A-C illustrate of a bone screw (10) that can be used in the orthopedic fixation device (100). The screw (10) includes a first end (12), a threaded intermediate portion (14), and second end in the form of a spherical 115 head (16). A spherical head allows for the polyaxial rotation of the tulip (70) about the screw head (16). The head (16) may have a roughened surface (18), which in some embodiments, may include threads that are parallel to each other. In preferred embodiments, the roughened surface (18) includes patterned grooves. Patterned grooves allow for increased locking strength. The grooves provide a deforming geometry to mate with the wedge for locking assembly angle. Those of ordinary skill in the art wilt appreciate that various other roughened surfaces may be used.

The top of the head (16) has a suitable configuration for mating with a corresponding driver, such as a tool engaging socket. As illustrated in FIG. 2B, in one embodiment, the top of the head has a hexalobular shaped indentation (600), which mates with the tip of a driver. The hexalobular head (600) creates a secure connection, preventing the driver from stripping the screw. The hexalobe design also increases surface contact and reduces wear on the screw driving bits.

The head (16) is preferably connected to an intermediate portion (14) by a neck relief (20) which allows for maximum amount of conical angulation of the tulip (70) about the bone screw head. The first end (12) preferably ends in a blunt tip (22), which provides safety for the user during implantation. The intermediate portion (14) preferably includes a dual lead thread (24), such as shown in FIG. 2C. The dual lead thread increases screw insertion rate, for faster implantation.

B. Coupling Element

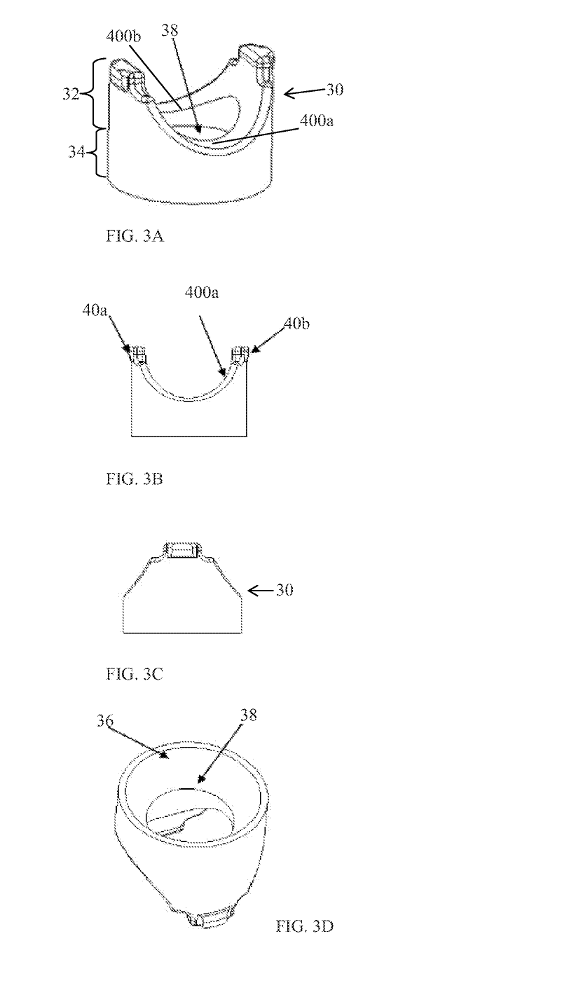

Referring to FIGS. 3A-D, the orthopedic fixation device includes a coupling wedge (30), which has an upper portion (32), a lower portion (34) and a thru hole (38). The coupling wedge is configured to be received completely by the tulip element (70).

The lower portion (34) of the coupling wedge (30) has an inner surface (36), which may be shaped as a concave approximately spherical or ball-shaped, segment. The inner surface (36) preferably forms a concave spherical cavity. The radius of the inner surface (36) may be larger than the radius of the enlarged head (16) of the screw body (10). Preferably, the radius of the inner surface (36) is approximately the same as the head (16) of the bone fastener (10). The inner surface (36) has a suitable size and shape to engage the top of the head (16) of the screw body (10). The inner surface (36) allows the bone screw head to slide relative to the surface, with conical angulation. The inner surface (36) generally matches the diameter of the top of the bone screw head, and provides a locking force on as much of the bone screw head as possible. In some embodiments, the inner surface (36) may be roughened. In an embodiment, the inner surface (36) may be threaded. In another embodiment, the inner surface (36) may be sandblasted. In yet another embodiment, the inner surface (36) may include a plurality of grooves. Those of ordinary skill in the art will appreciate that various other roughened surfaces may be used. In other embodiments, the inner surface (36) may be substantially smooth.

The upper portion (32) of the coupling wedge (30) contains two indentations (400a, 400b), one on each of the opposing sides of the wedge adapted for receiving the rod (300). The indentations (400a, 400b) are curved. such that each surrounds a portion of the cylindrical rod (300). The indentation (400) allows for the rod (300) to be centered in the tulip element (70), provides more surface area contact between the rod and the surface against which it locks compared to a straight, smooth surface, maximizing locking strength.

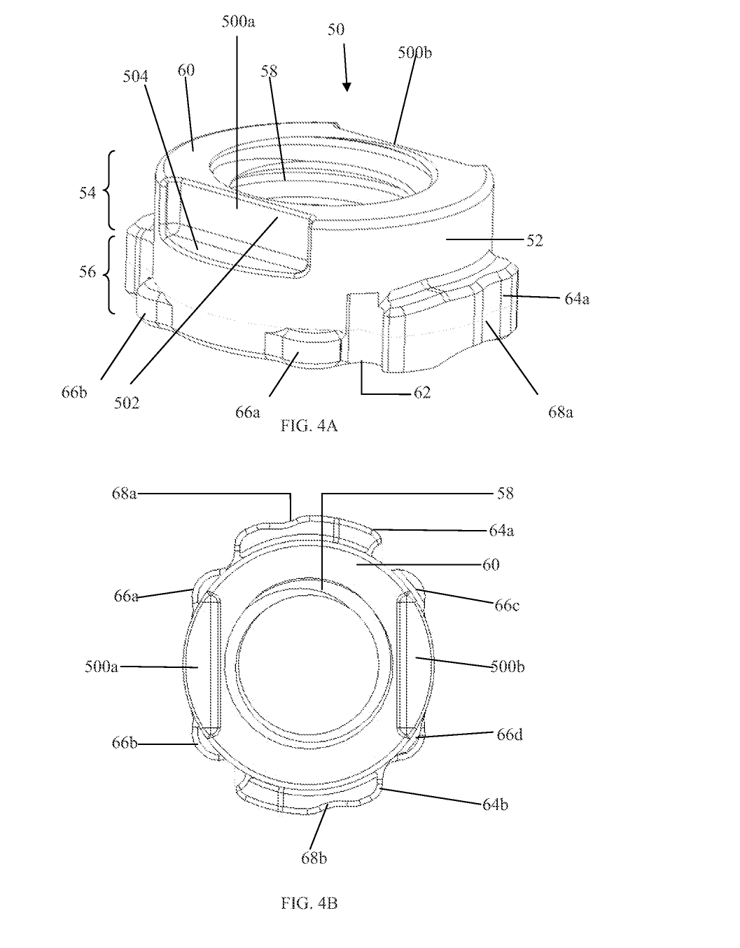

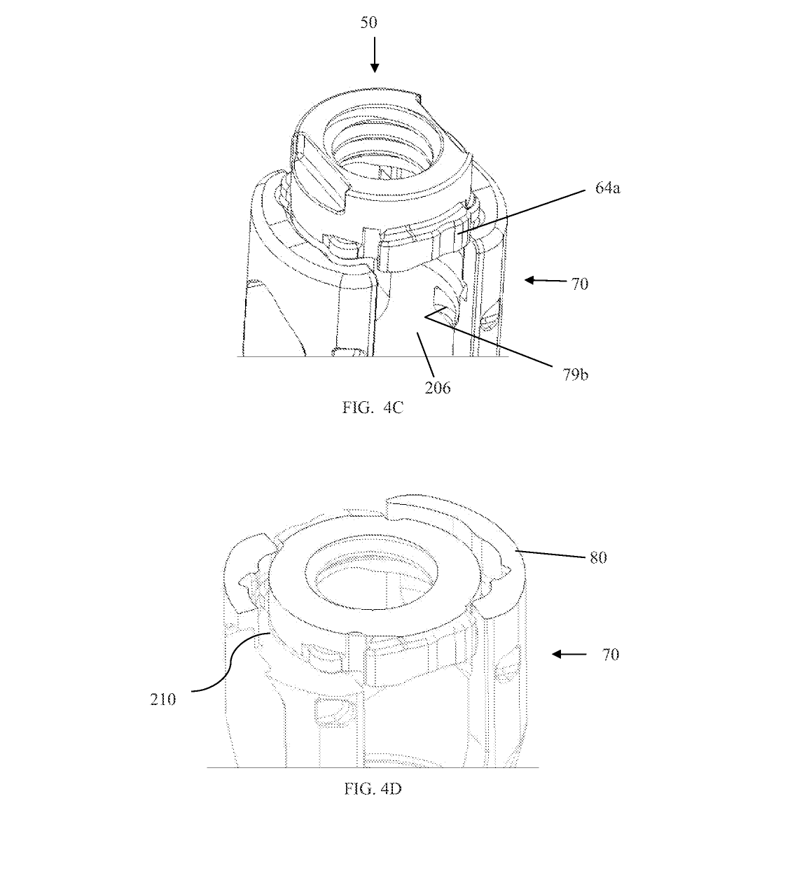

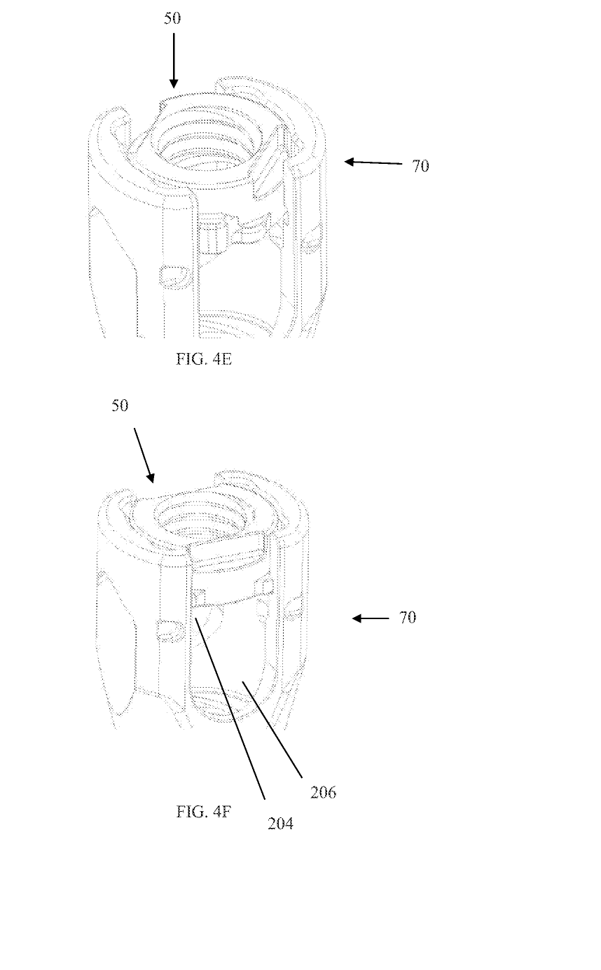

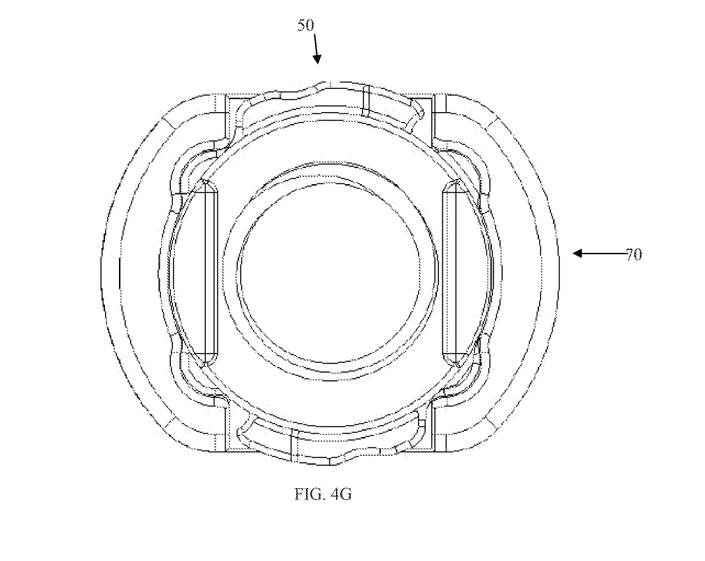

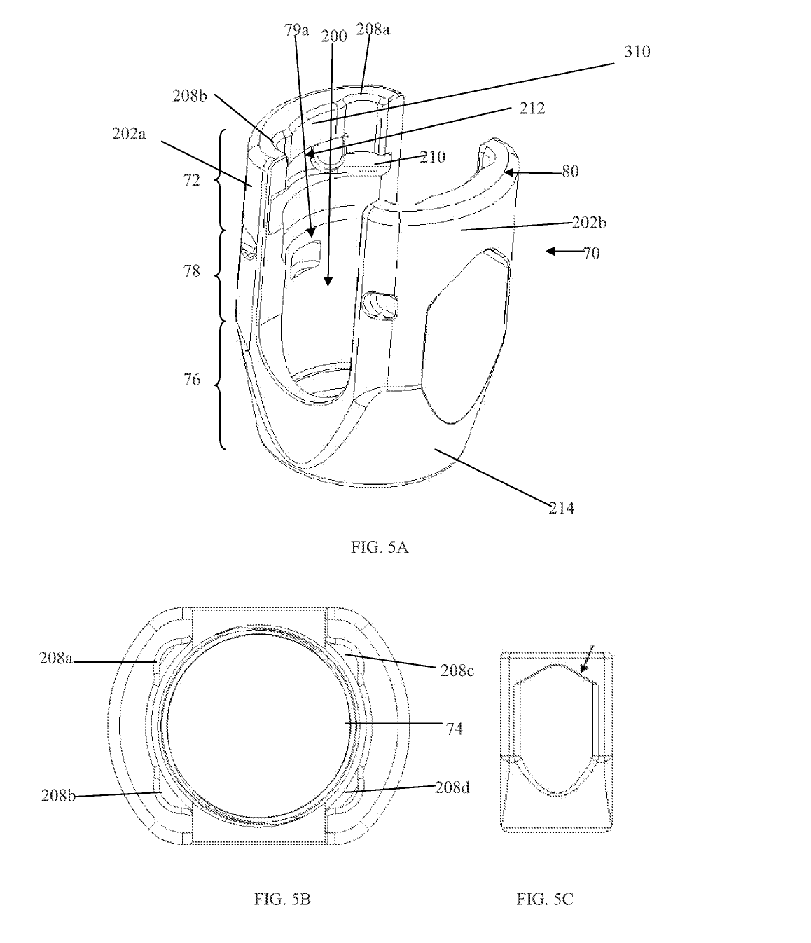

The upper portion (32) includes locking tabs (40a, 40b) at the top of the upper portion (32), configured to engage in corresponding coupling wedge grooves (79a, 79b) in the intermediate portion (78) of the tulip element (70) (See FIGS. 4C and 5A), thereby locking the coupling wedge in place, relative to the tulip element (70). The locking tabs (40a, 40b) provide positional orientation of the coupling wedge (30) in the tulip element (70). The inner surface (36) of the lower portion (34) is in direct contact with the bone screw head. The coupling wedge (30) provides a constant force on the screw head (16) to create polyaxial drag. Thus, the tulip will only move when oriented by the user or when a sufficient force (that can be moved by hand) is applied to change the orientation of the tulip element (70). The design provides sufficient resistance to hold the device in place but not enough to make the user work to move it. The angulation of the screw is preferably 30° or about 30° in all directions which makes a total conical angulation of 60 or about 60°.

C. Locking Cap

Referring to FIGS. 4A-4G, the coupling element includes a locking cap (50), which includes a generally cylindrical cap body (52). The cap body (52) has an upper portion (54) with an upper end surface (60), a lower portion (56) with a lower end surface (62) and an inner surface (58) defining a that hole. The inner surface (58) is configured to receive a locking set screw, and in some embodiments, has a bottom counterbore (92) that allows for the locking set screw (90) to be preassembled to the locking cap, and positions the set screw above the rod reduction clearance cut. The bottom counterbore (92) is a cylindrical relief that allows the set screw to seat fully into the locking cap and provides a flat surface against which the set screw can rest.

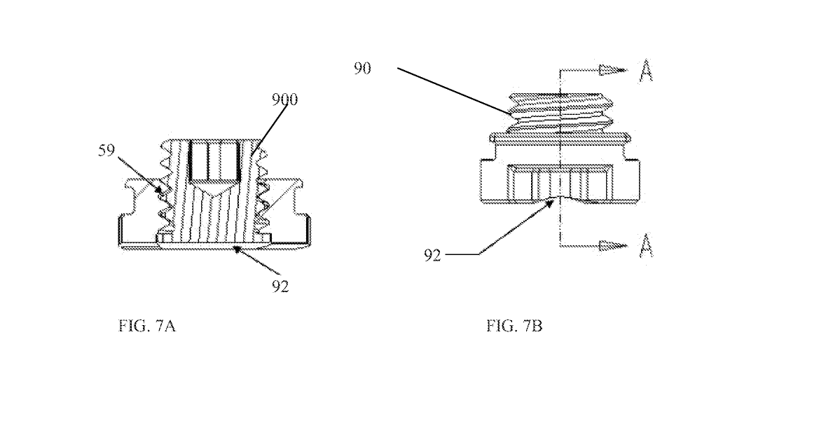

In a preferred embodiment, the inner surface (58) includes that cut threads (59) configured to mate corresponding threads (159) on the locking set screw (90). See FIG. 7A.

The upper end surface (60) of the cap body is configured to be received in the upper opening (74) of the tulip element (70) and to rest completely within the interior recess (200) of the tulip element, such that upper end surface (60) of the cap body aligns with the upper end (80) of the tulip element (70). Thus, when assembled, no portion of the cap body (e.g. shoulders or other tabs, flanges, or protrusions) extends beyond the upper end (80) of the tulip element (70). See FIG. 4F and 4G.

The locking cap (50) includes at least two radially spaced flanges (64) extending radially outward from the lower portion (56) of the cap body and in opposing relation to one another, and at least two radially spaced tabs (66) extending radially outward from the lower portion of the cap, and in opposing relation to one another. The radially spaced flanges (64a, 64b) include vertically oriented ridges (68a, 68b), preferably positioned in the middle of each flange (64).

The upper portion (54) of the locking cap element (50) includes driver attachment pockets (500) in opposing relation to one another, on opposing ends of the upper portion (54) of the locking cap element (50). The driver attachment pockets (500a, 500b) include a recess, formed by a first wall (502) extending downwards from the upper end surface (60) of the upper portion (54) of the cap body (56) and a second wall (504), extending outward in a perpendicular plane from the first wall (502) and away from the inner surface (58). The radially spaced tabs (66) are preferably located beneath the driver attachment pockets (500a, 500b).

The driver attachment pockets on the locking cap can have alternate configurations than depicted in FIGS. 4A-4C, for example, they could be rounded. The driver contains mating tabs configured to engage the driver attachment pockets. The distance between tabs in the driver is slightly smaller than the distance between pockets on the locking cap which creates a tension that allows the driver hold the locking cap (i.e. friction fit) while implanting it. The driver is preferably spring-loaded.

In another embodiment, the locking set screw can be preassembled into the locking cap, before placement of the cap into the tulip element (see FIG. 7A-B).

D. Tulip Element

An exemplary tulip element is illustrated in FIGS. 5A-5D. The tulip element (70) may include an upper portion (72) having an upper end (80) and an upper opening (74), an intermediate portion (78), and a lower portion (76) having a lower opening (82). The diameter of the upper opening (74) is typically greater than the diameter of the head (16) of the screw (10). This allows the screw to enter the tulip element (70) through the upper opening (74).

The upper portion (72) includes an interior recess (200) surrounded by two approximately partially cylindrically-shaped walls (202a, 202b), with slots (204) and (206) extending between the walls. The slots (204) and (206) are configured to allow for placement of the rod (300) into and through the tulip element.

The upper end (80) of each wall (202a, 202b) includes indentations or grooves (also referred to as a “bench tab grooves”) (208a-208d) having a suitable shape and size to mate with a tab (66) on the locking cap (50). The number and location of the grooves (208a-208d) corresponds to the number and location of the tabs (66a-66d) on the locking cap (50). Preferably the groves are semicircular in shape.

The upper portion (72) of each wall (202a, 202b) also has a second set of grooves (210) which serve as bench tab tracks, through which the bench tabs exit the tulip. The bench tab tracks (210) extend inwardly from either side of the interior (310) of each wall (202a, 202b) and are separated by a third set of grooves (212), configured to receive locking flanges (64a, 64b) on the locking cap (50). The third set of grooves (212) extends inwardly into the interior wall (310), and is located below the bench tab groove (208a-208d).

The intermediate portion (78) of each wall (202a, 202b) also has an indentation or groove (also referred to as a “wedge groove”) (79) adapted to receive, orient, and lock the coupling wedge (30). The wedge grooves (79a, 79b) receive the coupling wedge locking tabs (40a, 40b), which snap into the wedge grooves, and thereby maintain axial and angular position for the wedge.

The lower portion (76) of the tulip element (70) has a tapered bottom edge (214) which allows for close positioning of the tulip to bone, by minimizing the size so that the tulip can sit in pockets in the bone. The tapered reduction geometry is shown in FIG. 5A. The lower portion (76) contains a lower opening or thru hole (82), with a lip, which allows for assembly of the bone screw, and provides a seat for the bone screw head, where the conical angulation pivots.

### II. Use of the Orthopedic Fixation Device

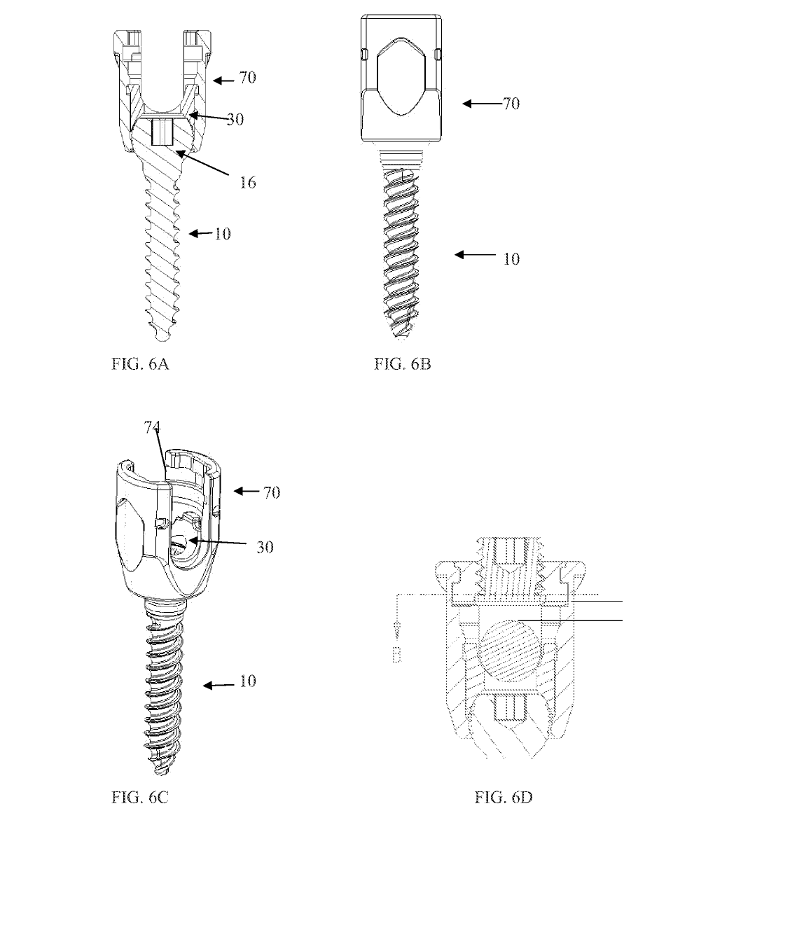

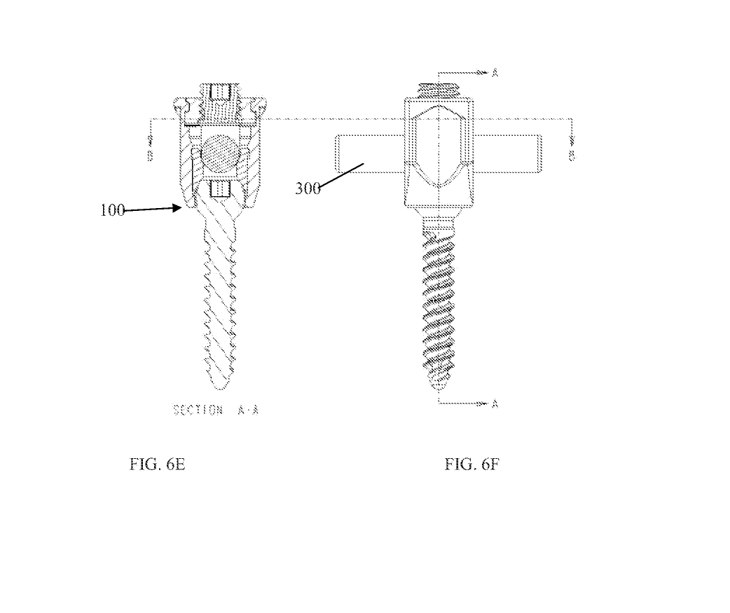

FIG. 6A-G shows the elements of the orthopedic device in various states of assembly to form the complete fixation device. FIG. 6A-C illustrates different views of the assembled screw, wedge and tulip element. The tulip element (70) completely houses the coupling wedge (30) and the screw head (16) of the bone screw (10).

The bone screw (10) is inserted from the upper opening (74) of the tulip element (70), until the screw head (16) mates with the spherical seat at the bottom of the tulip element. The head of the bone screw (10) is retained within the tulip and proximate to the second and lower end of the tulip, with the threaded shaft extending out of the tulip through the second opening (82) thereof.

Then the coupling wedge (30) is inserted into the tulip element (70) through the upper opening (74), until the wedge locking tabs (40a, 40b) align with the retaining wedge grooves (79a, 79b) in the tulip element (70). The coupling wedge (30) is pressed into the tulip element (70) from the top, until it clicks into position in the wedge grooves (79a, 79b). In this position, the inner surface (36) of the coupling wedge (30), engages the head (16) of the bone screw (10).

Appropriate placement of the bone screw or fastener and coupling wedge in the tulip element provides sufficient clearance for a rod through the slots (204 and 206) in the walls of the tulip element.

FIGS. 4C-G illustrates how the locking cap is turned when it is in the tulip element to reach the locked position. As shown in FIG. 4C, when in use, the bench tabs (66) of the locking cap (50) engage the tulip element (70) at the same time as the flanges (64a, 64b), with the bench tabs (66a-66d) in contact with the corresponding groove (208a-208d) in the tulip (70) and one of the flanges (64a, 64b) in each of the slots (204 and 206). The cap is locked via a 90° turn, such that the bench tabs exit the tulip. In alternative embodiments, the cap is turned a different arc length until the flanges reach the corresponding groove to reach the locked position.

FIG. 4D shows a partial cut-a-way section of the locking cap (50) with the bench tabs (66) on the tulip bench tab tracks (210).

FIG. 4E shows the locking cap (50) in the midst of being rotated to the locked position.

FIGS. 4F and 4G show the locking cap and tulip with the locking cap in the locked and unlocked positions, respectively. In the locked position, the flanges (64a, 64b) are received in the tulip grooves (212). In the locked position, the locking cap tabs (66) and the driver attachment pockets (200) typically exit the walls (202a, 202b) of the tulip (70) and rest in the slots (204 and 206) extending between the walls (see FIG. 4F).

Once the locking cap is captured in the tulip element and is in the locked position, the set screw (90) is then turned into the locking cap (50) to lock the pedicle screw in its desired position (e.g. to secure the angle of the pedicle screw) and to lock the rod to the screw. The cap set screw (90) distributes the load through the rod and coupling wedge onto the head of the bone screw.

In another embodiment, the locking set screw can be preassembled into the locking cap, before placement of the cap into the tulip element (see FIG. 7A-B).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

I claim:

1. An orthopedic fixation device comprising:
(i) a bone fastener comprising a threaded shaft and a head at a proximal end of the shaft; and
(ii) a coupling element, wherein the coupling element comprises:
(a) a coupling wedge,
(b) a locking cap, and
(c) a tulip element, wherein the tulip element is configured to receive all of the coupling wedge and the locking cap.

2. The orthopedic fixation device of claim 1, wherein the tulip element comprises:
(a) outer and inner walls defining a first and upper open end and a second and lower open end, and
(b) opposing first and second slots extending from the first end towards the second end.

3. The orthopedic fixation device of claim 2, wherein the tulip element further comprises an upper portion, wherein the upper portion comprises a plurality of grooves on the inner walls configured to receive the locking cap.

4. The orthopedic fixation device of claim 2, wherein the tulip element further comprises an intermediate portion, wherein the intermediate portion comprises a. first set of grooves on the inner walls configured to receive the coupling wedge.

5. The orthopedic fixation device of claim 1, wherein the lower end of the tulip element comprises an outer surface with a tapered reduction geometry, wherein the diameter of the outer surface decreases moving from the top of the lower end to the bottom of the lower end.

6. The orthopedic fixation device of claim 2, wherein the coupling wedge comprises an upper end and a tower end, wherein the lower end of the coupling wedge is configured with a tapered lower surface for receiving the lower end of the tulip element.

7. The orthopedic device of claim 1, wherein the coupling wedge comprises an indentation configured fir receiving a rod.

8. The orthopedic device of claim 1, wherein the coupling wedge further comprises outwardly extending tabs configured to mate with one or more grooves in the tulip.

9. The orthopedic fixation device of claim 1, wherein the cap comprises a cap body, an upper portion, a lower portion, and one or more driver attachment pockets.

10. The orthopedic fixation device of claim 9, wherein the cap further comprises
(i) a plurality of radially spaced flanges extending radially outward from the lower portion of the cap body and in opposing relation to one another, and
(ii) a plurality of radially spaced tabs extending radially outward from the lower portion of the cap and in opposing relation to one another.

11. The orthopedic fixation device of claim 10, wherein the radially protruding flanges include vertically-oriented ridges, and
wherein the tabs are located on the lower portion of the cap body, and below the driver pocket attachments.

12. The orthopedic device of claim 10, wherein the tulip element further comprises a first set of grooves formed on the interior sidewalk of the tulip for receiving the cap flanges, and tracks for receiving the cap tabs.
